(19) United States
(12) Patent Application Publication
(10) Pub. No.: US 2022/0002815 A1
(43) Pub. Date: Jan. 6, 2022

(54) METHODS AND GENOMIC CLASSIFIERS FOR PROGNOSIS OF BREAST CANCER AND PREDICTING BENEFIT FROM ADJUVANT RADIOTHERAPY

(71) Applicant: PFS Genomics, Inc., Madison, WI (US)

(72) Inventors: S. Laura Chang, Ann Arbor, MI (US); Lori J Pierce, Ann Arbor, MI (US); Corey Speers, Saline, MI (US); Felix Feng, Hillsborough, CA (US); Per Malmström, Skanör (SE); Mårten Fernö, Limhamn (SE); Erik Holmberg, Asperö (SE); Per O. Karlsson, Gothenburg (SE)

(73) Assignee: PFS Genomics, Inc., Madison, WI (US)

(21) Appl. No.: 17/290,759

(22) PCT Filed: Nov. 4, 2019
(86) PCT No.: PCT/IB2019/001181

§ 371 Date: May 2, 2021

Related U.S. Application Data
(60) Provisional application No. 62/755,546, filed on Nov. 4, 2018.

(51) Int. Cl. C12Q 1/6886; G16B 25/10

(52) U.S. Cl. CPC C12Q 1/6886; G16B 25/10; C12Q 2600/158; C12Q 2600/118; C12Q 2600/106

(57) ABSTRACT

Methods, systems, and kits for the diagnosis, prognosis, and treatment of breast cancer in a subject are disclosed. The invention also provides biomarkers and clinically useful genomic classifiers for identifying subjects at low risk of breast cancer recurrence who are likely to benefit from adjuvant radiotherapy. Further disclosed herein, in certain instances, are probe sets for use in detecting such biomarkers for determining the risk of breast cancer recurrence in a subject (e.g., locoregional recurrence). Methods of treating breast cancer based on expression profiling to determine the risk of breast cancer recurrence are also provided.

### RELATED APPLICATIONS

This application claims priority to U.S. Provisional Patent Application Ser. No. 62/755,546, filed on Nov. 4, 2018, which is hereby incorporated by reference herein in its entirety.

### FIELD OF THE INVENTION

The present invention pertains to the field of personalized medicine and methods for treating breast cancer. In particular, the invention relates to the use of transcriptomic profiling for prognosis of cancer recurrence (e.g., locoregional recurrence) in subjects with breast cancer and methods for identifying subjects who are likely to benefit from radiotherapy.

### BACKGROUND OF THE INVENTION

Cancer is the uncontrolled growth of abnormal cells anywhere in a body. The abnormal cells are termed cancer cells, malignant cells, or tumor cells. Many cancers and the abnormal cells that compose the cancer tissue are further identified by the name of the tissue that the abnormal cells originated from (for example, breast cancer). Cancer cells can proliferate uncontrollably and form a mass of cancer cells. Cancer cells can break away from this original mass of cells, travel through the blood and lymph systems, and lodge in other organs where they can again repeat the uncontrolled growth cycle. This process of cancer cells leaving an area and growing in another body area is often termed metastatic spread or metastatic disease. For example, if breast cancer cells spread to a bone (or anywhere else), it can mean that the individual has metastatic breast cancer.

Most women with early-stage invasive breast cancer are operated with breast-conserving surgery (BCS) and adjuvant post-operative radiation therapy (RT). While RT provides a significant reduction in local recurrence risk and increased breast cancer-specific survival, better treatment selection is needed. Meta-analyses from the Early Breast Cancer Trialists' Collaborative Group have shown that at 10 years post-surgery, 70% of women with stage I-II node-negative breast cancer are free from local recurrence despite not receiving RT, and 10% experience a local recurrence even with the receipt of RT, thus suggesting both over- and under treatment. (Darby S et al. Lancet 2011; 378(9804): 1707-16.)

Several clinico-pathologic risk factors have been described for local recurrence, where young age is one of the strongest, and younger patients are commonly given a radiation boost. (Fredriksson I et al. The British journal of surgery 2003; 90(9): 1093-102 and Fredriksson I et al. European journal of cancer (Oxford, England: 1990) 2001; 37(12): 1537-44.) However, with more modern surgical and oncological treatment, the rate of local recurrence has considerably decreased, attributed in part to better surgery and better adjuvant treatment. (Bouganim N et al. Breast cancer research and treatment 2013; 139(2): 603-6 and Aalders K C et al. European journal of cancer (Oxford, England: 1990) 2016; 63: 118-26.) This has led to several trials aimed at de-escalating RT in presumed low-risk women, including the PRIMETIME, LUMINA, and NCT02653755 trials. (Wickberg A et al. European journal of surgical oncology: the journal of the European Society of Surgical Oncology and the British Association of Surgical Oncology 2018; 44(7): 951-6 and Kirwan C C et al. Clinical oncology (Royal College of Radiologists (Great Britain)) 2016; 28(9): 594-6.) However, there remains a need for improved methods for identifying breast cancer subjects that will benefit from radiotherapy. Further, there is a need for novel gene signatures that are prognostic for loco-regional recurrence and radiation sensitivity.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates to methods, systems, and kits for the diagnosis, prognosis, and treatment of breast cancer in a subject. The invention also provides biomarkers and classifiers for identifying subjects at low risk of breast cancer recurrence and likely to benefit from adjuvant radiotherapy. Further disclosed herein, in certain instances, are probe sets for use in detecting such biomarkers for determining the risk of breast cancer recurrence in a subject. The invention further provides biomarkers and classifiers for identifying subjects at risk for locoregional recurrence (LRR) and predicting response to radiotherapy. Methods of treating breast cancer based on expression profiling and/or age to determine the risk of breast cancer recurrence are also provided.

In some embodiments, the invention provides a method for prognosing and/or predicting benefit from adjuvant radiotherapy in a subject having breast cancer, the method comprising: a) obtaining or having obtained an expression level in a sample from a subject for a plurality of genes, wherein the plurality of genes are selected from Table 2; and b) determining that the subject is at low risk or not at low risk of cancer recurrence based on the expression level, and/or likely to benefit from adjuvant radiotherapy based on the expression level, thereby prognosing and/or predicting benefit from adjuvant radiotherapy in the subject. In some embodiments, the method further comprises administering adjuvant radiotherapy therapy if the subject is identified as likely to benefit from adjuvant radiotherapy, and recommending treatment intensification if the subject is not identified as likely to benefit from adjuvant radiotherapy. In other embodiments, the cancer recurrence is local or locoregional recurrence or distant recurrence (metastasis). In some embodiments, the levels of expression of one or more of the genes selected from Table 2 may be increased or decreased compared to a control. In some embodiments, the expression levels of all of the genes selected from Table 2 are measured in the biological sample. In some embodiments, the method further comprises treating the subject with adjuvant radiotherapy. In still other embodiments, the method further comprises treating the subject with mastectomy, radiation boost, or adjuvant systemic therapy. In some embodiments, the subject is treated with radiotherapy following breast conserving surgery (BCS). In some embodiments, the method further comprises determining that the subject is at low risk of cancer recurrence based on the age of the subject, or determining that the subject is not at low risk of cancer recurrence based on the age of the subject. In some embodiments, the cancer recurrence is local or locoregional recurrence or distant recurrence (metastasis).

In some embodiments, the invention provides a method comprising: a) obtaining or having obtained an expression level in a sample from a subject for a plurality of genes, wherein the plurality of genes are selected from Table 2; and b) determining that the subject is at low risk of cancer recurrence and likely to benefit from treatment with adjuvant radiotherapy based on the expression level, or determining that the subject is not at low risk of cancer recurrence and likely to require treatment intensification based on the expression level. In other embodiments, the method further comprises administering adjuvant radiotherapy therapy if the subject is identified as likely to benefit from adjuvant radiotherapy, and recommending treatment intensification if the subject is not identified as likely to benefit from adjuvant radiotherapy. In other embodiments, the cancer recurrence is local or locoregional recurrence or distant recurrence (metastasis). In some embodiments, the levels of expression of one or more of the genes selected from Table 2 may be increased or decreased compared to a control. In some embodiments, the expression levels of all of the genes selected from Table 2 are measured in the biological sample. In still other embodiments, the method further comprises treating the subject with mastectomy, radiation boost, or adjuvant systemic therapy. In some embodiments, the subject is treated with radiotherapy following breast conserving surgery (BCS). In some embodiments, the method further comprises determining that the subject is at low risk of cancer recurrence based on the age of the subject, or determining that the subject is not at low risk of cancer recurrence based on the age of the subject. In some embodiments, the cancer recurrence is local or locoregional recurrence or distant recurrence (metastasis).

In some embodiments, the invention provides a method of treating breast cancer in a subject, comprising: a) obtaining or having obtained an expression level in a sample from a subject for a plurality of genes, wherein the plurality of genes are selected from Table 2; b) determining that the subject is at low risk of cancer recurrence and likely to benefit from treatment with adjuvant radiotherapy based on the expression level, or determining that the subject is not at low risk of cancer recurrence and likely to require treatment intensification based on the expression level; and c) administering adjuvant radiotherapy therapy if the subject is identified as likely to benefit from adjuvant radiotherapy based on the expression level, and recommending treatment intensification if the subject is not identified as likely to benefit from adjuvant radiotherapy based on the expression level. In other embodiments, the cancer recurrence is local or locoregional recurrence or distant recurrence (metastasis). In some embodiments, the levels of expression of one or more of the genes selected from Table 2 may be increased or decreased compared to a control. In some embodiments, the expression levels of all of the genes selected from Table 2 are measured in the biological sample. In still other embodiments, the method further comprises treating the subject with mastectomy, radiation boost, or adjuvant systemic therapy. In some embodiments, the subject is treated with radiotherapy following breast conserving surgery (BCS). In some embodiments, the method further comprises determining that the subject is at low risk of cancer recurrence based on the age of the subject, or determining that the subject is not at low risk of cancer recurrence based on the age of the subject. In some embodiments, the cancer recurrence is local or locoregional recurrence or distant recurrence (metastasis).

In some embodiments, the plurality of genes used in the methods and genomic classifiers of the present invention are selected from the group consisting of ATP binding cassette subfamily C member 8 (ABCC8), BTG anti-proliferation factor 3 (BTG3), cyclin B1 (CCNB1), centromere protein F (CENPF), creatine kinase mitochondrial 1B (CKMT1B), cornichon family AMPA receptor auxiliary protein 4 (CNIH4), cellular retinoic acid binding protein 2 (CRABP2), enoyl-CoA hydratase domain containing 2 (ECHDC2), eukaryotic elongation factor 2 kinase (EEF2K), eukaryotic translation initiation factor 3 subunit L (EIF3L), ectonucleoside triphosphate diphosphohydrolase 6 (putative) (ENTPD6), epoxide hydrolase 2 (EPHX2), H2A histone family member Z (H2AFZ), hydroxysteroid 17-beta dehydrogenase 4 (HSD17B4), karyopherin subunit alpha 2 (KPNA2), lymphoid enhancer binding factor 1 (LEF1), NEDD4 binding protein 2 like 1 (N4BP2L1), NIMA related kinase 10 (NEK10), prefoldin subunit 4 (PFDN4), pleckstrin and Sec7 domain containing 3 (PSD3), runt related transcription factor 1 (RUNX1), SEC14 like lipid binding 2 (SEC14L2), serine/threonine kinase 39 (STK39), TBC1 domain family member 8 (TBC1D8), thymosin beta 15a (TMSB15A), USO1 vesicle transport factor (USO1), and zinc finger and BTB domain containing 16 (ZBTB16).

In certain embodiments, the subject has estrogen receptor positive (ER+) breast cancer, human epidermal growth factor receptor 2 negative (HER2−) breast cancer, Stage I-II breast cancer, or node-negative breast cancer and/or is post-menopausal.

In other embodiments, the biological sample obtained from the subject is a breast biopsy or tumor sample. In yet other embodiments, biological sample is a bodily fluid or tissue of the subject that contains breast cancer cells. In certain embodiments, nucleic acids (e.g., RNA transcripts) comprising sequences from genes selected from Table 2, or complements thereof, are further isolated from the biological sample, and/or purified, and/or amplified prior to analysis.

In other embodiments, the expression levels of biomarkers are determined by in situ hybridization, PCR-based methods, array-based methods, immunohistochemical methods, RNA assay methods, or immunoassay methods. In other embodiments, the levels of gene expression are determined using one or more reagents. In certain embodiments, the one or more reagents are nucleic acid probes, nucleic acid primers, and/or antibodies. In other embodiments, determining the level of expression of a biomarker comprises measuring the level of a nucleic acid (e.g., RNA transcript).

In some embodiments, the level of expression of at least one gene is reduced compared to a control. In other embodiments, the level of expression of at least one gene is increased compared to a control.

In certain embodiments, the methods described herein are performed prior to treatment of the subject with adjuvant radiotherapy. In certain embodiments, the methods described herein are performed prior to treatment of the subject with mastectomy, radiation boost, or adjuvant systemic therapy.

In other embodiments, the method further comprises calculating a risk score for the subject, wherein adjuvant radiotherapy is administered to the subject if the subject is identified as being at low risk of cancer recurrence and likely to benefit from adjuvant radiotherapy based on both the risk score and the expression levels of the genes selected from Table 2 in the biological sample, and recommending treatment intensification to the subject if the subject is not identified as being likely to benefit from adjuvant radiotherapy based on both the risk score and the expression levels of the genes selected from Table 2 in the biological sample. In some embodiments, the cancer recurrence is local or locoregional recurrence or distant recurrence (metastasis).

The significance of the expression levels of one or more biomarker genes may be evaluated using, for example, a T-test, P-value, KS (Kolmogorov Smirnov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the expression level of the one or more targets may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) or Multivariable Analysis Hazard Ratio P-value (mvaHRPval).

In another aspect, the invention includes a probe set for determining a prognosis of a subject having breast cancer and whether or not to treat the subject with radiotherapy, the probe set comprising a plurality of probes for detecting a plurality of target nucleic acids, wherein the plurality of target nucleic acids comprises one or more gene sequences, or complements thereof, of genes selected from Table 2. Probes may be detectably labeled to facilitate detection. In some embodiments, the prognosis comprises cancer recurrence prognosis. In further embodiments, the cancer recurrence is local or locoregional recurrence or distant recurrence (metastasis).

In another aspect, the invention includes a system for determining a prognosis of a subject who has breast cancer and whether or not to treat the subject with radiotherapy, the system comprising: a) a probe set described herein; and b) a computer model or algorithm for analyzing an expression level or expression profile of the plurality of target nucleic acids hybridized to the plurality of probes in a biological sample from a subject who has breast cancer and determining if the subject is at low risk of cancer recurrence based on the expression level or expression profile and should be treated with radiotherapy. In some embodiments, the cancer recurrence is local or locoregional recurrence or distant recurrence (metastasis).

In some embodiments, the invention includes a kit for determining a prognosis of a subject having breast cancer and whether or not to treat the subject with adjuvant radiotherapy, the kit comprising agents for measuring levels of expression of a plurality of genes selected from Table 2. In other embodiments, the kit may include one or more agents (e.g., hybridization probes, PCR primers, or microarray) for measuring levels of expression of a plurality of genes, wherein said plurality of genes comprises one or more genes selected from Table 2, a container for holding a biological sample comprising breast cancer cells isolated from a human subject for testing, and printed instructions for reacting the agents with the biological sample or a portion of the biological sample to determine if the subject is at low risk of cancer recurrence of the breast cancer and likely to benefit from treatment with adjuvant radiotherapy. In some embodiments, the cancer recurrence is local or locoregional recurrence or distant recurrence (metastasis). In other embodiments, the agents are packaged in separate containers. In yet other embodiments, the kit further comprises one or more control reference samples or other reagents for measuring gene expression (e.g., reagents for performing PCR, RT-PCR, microarray analysis, a Northern blot, an immunoassay, or immunohistochemistry). In yet other embodiments, the kit comprises agents for measuring the levels of expression of a plurality of genes listed in Table 2. In another embodiment, the kit comprises agents for measuring the levels of expression of all the genes listed in Table 2. In certain embodiments, the kit comprises a probe set, as described herein, for detecting a plurality of target nucleic acids, wherein the plurality of target nucleic acids comprises one or more gene sequences, or complements thereof, of genes selected from Table 2, or any combination thereof.

In other embodiments, the kit further comprises a system, wherein the system comprises: a) a probe set comprising a plurality of probes for detecting a plurality of target nucleic acids, wherein the plurality of target nucleic acids comprises one or more gene sequences, or complements thereof, of genes selected from Table 2; and b) a computer model or algorithm for analyzing an expression level or expression profile of the plurality of target nucleic acids hybridized to the plurality of probes in a biological sample from a subject who has breast cancer and determining if the subject is at low risk of cancer recurrence based on the expression level or expression profile and likely to benefit from treatment with adjuvant radiotherapy. In some embodiments, the cancer recurrence is local or locoregional recurrence or distant recurrence (metastasis).

These and other embodiments of the subject invention will readily occur to those of skill in the art in view of the disclosure herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 shows an evaluation of previously reported signature prognostic for locoregional recurrence and/or treatment predictive for adjuvant radio therapy. Signatures were evaluated with Cox proportional hazards modelling in the full cohort, in the RT arm and in the no RT arm. For prognostication, patients were split by the 75th percentile score with the respective signatures. Interaction between RT and signatures were made with the raw continuous signature scores.

FIGS. 2A-2D show performance of a novel classifier for prognostication of locoregional recurrence and treatment prediction for adjuvant radio therapy. FIG. 2A shows survival analysis for high and low classifier scores (as split by the 75th percentile score) in the full cohort. FIG. 2B shows radio therapy benefit in patients classified as low risk by the novel classifier. FIG. 2C shows radiotherapy benefit in patients classified as high risk with the novel classifier. FIG. 2D shows interaction of radio therapy and classifier scores. Continuous classifier scores are presented with the risk for locoregional recurrence with or without radio therapy.

FIGS. 3A and 3B show prognostic performance of the novel classifier with or without radio therapy and survival analysis for patients split by the 75th percentile score.

FIG. 4 shows Kaplan-Meier survival analysis for classifier high vs low scores and RT benefit in patients classified as high or low risk for previously reported signatures.

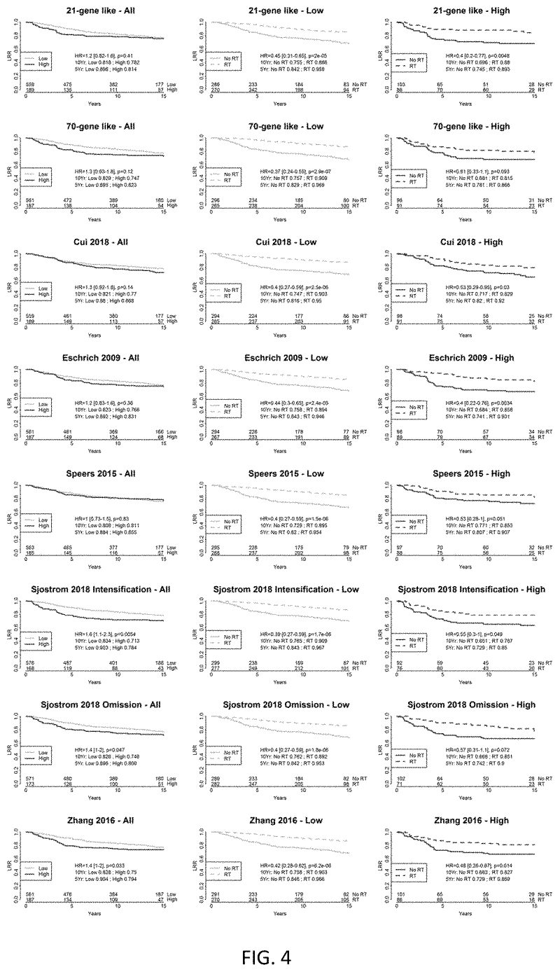

FIGS. 5A-5H shows interaction analysis for previously reported signatures.

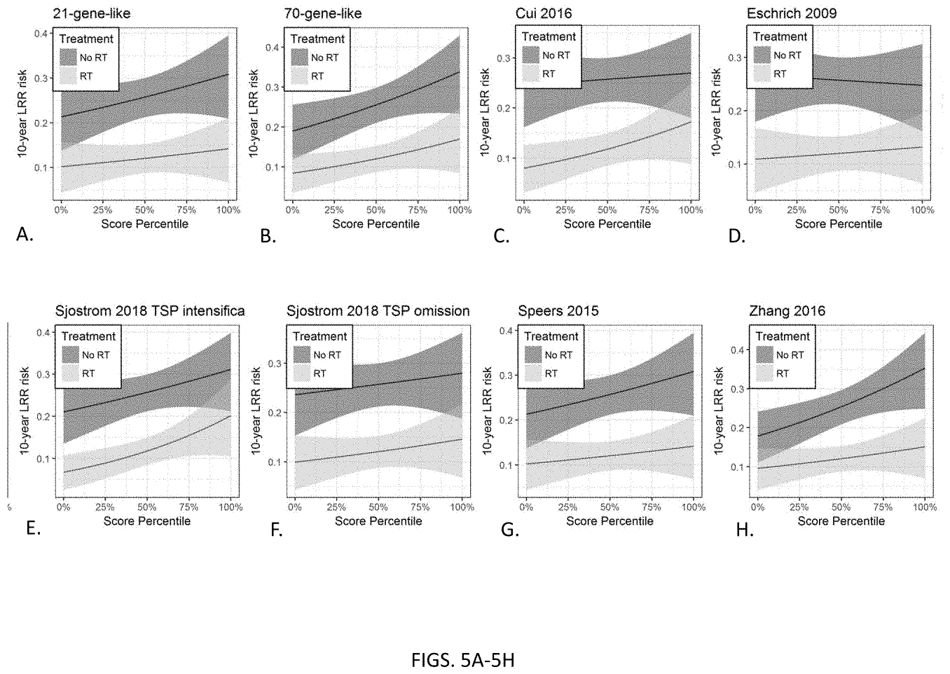

### DETAILED DESCRIPTION OF THE INVENTION

The present invention discloses systems and methods for diagnosing, predicting, and/or monitoring the status or outcome of a breast cancer in a subject using expression-based analysis of a plurality of gene targets. Generally, the method comprises (a) obtaining or having obtained an expression level in a sample from a subject for a plurality of genes; (b) determining that the subject is at risk of cancer recurrence based on the expression level of the plurality of genes; and c) administering or withholding adjuvant radiotherapy therapy if the subject is identified as being at risk of cancer recurrence based on the expression level of the plurality of gene targets.

Assaying the expression level for a plurality of gene targets in the sample may comprise applying the sample to a microarray. In some instances, assaying the expression level may comprise the use of an algorithm. The algorithm may be used to produce a genomic classifier. Alternatively, the classifier may comprise a probe selection region. In some instances, assaying the expression level for a plurality of targets comprises detecting and/or quantifying the plurality of targets. In some embodiments, assaying the expression level for a plurality of targets comprises sequencing the plurality of targets. In some embodiments, assaying the expression level for a plurality of targets comprises amplifying the plurality of targets. In some embodiments, assaying the expression level for a plurality of targets comprises quantifying the plurality of targets. In some embodiments, assaying the expression level for a plurality of targets comprises conducting a multiplexed reaction on the plurality of targets.

Further disclosed herein are methods for determining if the subject is at low risk of recurrence of the breast cancer. Generally, the method comprises: (a) providing a sample comprising breast cancer cells from a subject; (b) assaying the expression level for a plurality of targets in the sample; and (c) determining if the subject is at low risk of recurrence of the breast cancer based on the expression level of the plurality of targets and whether or not to treat the subject with adjuvant radiotherapy, chemotherapy, or endocrine therapy. For example, a subject identified as being at low risk of recurrence of the breast cancer according to the methods of the present invention, may be more likely to respond to adjuvant radiotherapy, whereas a subject identified as being at higher risk of recurrence of the breast cancer may be less likely to respond to adjuvant radiotherapy.

Before the present invention is described in further detail, it is to be understood that this invention is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present invention.

### Targets

In some instances, assaying the expression level of a plurality of genes comprises detecting and/or quantifying a plurality of target analytes. In some embodiments, assaying the expression level of a plurality of genes comprises sequencing a plurality of target nucleic acids. In some embodiments, assaying the expression level of a plurality of biomarker genes comprises amplifying a plurality of target nucleic acids. In some embodiments, assaying the expression level of a plurality of biomarker genes comprises conducting a multiplexed reaction on a plurality of target analytes.

The methods disclosed herein often comprise assaying the expression level of a plurality of targets. The plurality of targets may comprise coding targets and/or non-coding targets of a protein-coding gene or a non-protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non-protein-coding gene structure may comprise an exon and intron. Usually, the exon region of a non-protein-coding gene primarily contains a UTR. The non-protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of targets comprises one or more targets selected from Table 2. In some instances, the plurality of targets comprises at least about 2, at least about 3, at least about 4, at least about 5, at least about 6, at least about 7, at least about 8, at least about 9, at least about 10, at least about 15, at least about 20, at least about 25, or at least about 27 targets selected from Table 2.

In some embodiments, the plurality of targets are selected from the group consisting of ATP binding cassette subfamily C member 8 (ABCC8), BTG anti-proliferation factor 3 (BTG3), cyclin B1 (CCNB1), centromere protein F (CENPF), creatine kinase mitochondrial 1B (CKMT1B), cornichon family AMPA receptor auxiliary protein 4 (CNIH4), cellular retinoic acid binding protein 2 (CRABP2), enoyl-CoA hydratase domain containing 2 (ECHDC2), eukaryotic elongation factor 2 kinase (EEF2K), eukaryotic translation initiation factor 3 subunit L (EIF3L), ectonucleoside triphosphate diphosphohydrolase 6 (putative) (ENTPD6), epoxide hydrolase 2 (EPHX2), H2A histone family member Z (H2AFZ), hydroxysteroid 17-beta dehydrogenase 4 (HSD17B4), karyopherin subunit alpha 2 (KPNA2), lymphoid enhancer binding factor 1 (LEF1), NEDD4 binding protein 2 like 1 (N4BP2L1), NIMA related kinase 10 (NEK10), prefoldin subunit 4 (PFDN4), pleckstrin and Sec7 domain containing 3 (PSD3), runt related transcription factor 1 (RUNX1), SEC14 like lipid binding 2 (SEC14L2), serine/threonine kinase 39 (STK39), TBC1 domain family member 8 (TBC1D8), thymosin beta 15a (TMSB15A), USO1 vesicle transport factor (USO1), and zinc finger and BTB domain containing 16 (ZBTB16).

In some instances, the plurality of targets comprises a coding target, non-coding target, or any combination thereof. In some instances, the coding target comprises an exonic sequence. In other instances, the non-coding target comprises a non-exonic or exonic sequence. Alternatively, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non-protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

The plurality of targets may comprise one or more targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), linear model, or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of targets may comprise one or more targets selected from a linear model classifier. The plurality of targets may comprise two or more targets selected from a linear model classifier. The plurality of targets may comprise three or more targets selected from a linear model classifier. The plurality of targets may comprise 2, 3, 4, 5, 6, 7, 8, 9, 10, 11, 12, 13, 14, 15, 20, 25, 27 or more targets selected from a linear model classifier. The linear model classifier may be an LM2, and LM3, or an LM4 classifier. The linear model classifier may be an LM27 classifier (e.g., a linear model classifier with 27 targets). For example, a linear model classifier of the present invention may comprise two or more targets selected from Table 2.

The plurality of targets may comprise one or more targets selected from an SVM classifier. The plurality of targets may comprise 2, 3, 4, 5, 6, 7, 8, 9, 10 or more targets selected from an SVM classifier. The plurality of targets may comprise 12, 13, 14, 15, 17, 20, 22, 25 or more targets selected from an SVM classifier. The SVM classifier may be an SVM2 classifier. An SVM classifier of the present invention may comprise two or more targets selected from Table 2.

The plurality of targets may comprise one or more targets selected from a KNN classifier. The plurality of targets may comprise 2, 3, 4, 5, 6, 7, 8, 9, 10 or more targets selected from a KNN classifier. The plurality of targets may comprise 12, 13, 14, 15, 17, 20, 22, 25 or more targets selected from a KNN classifier. For example, a KNN classifier of the present invention may comprise two or more targets selected from Table 2.

The plurality of targets may comprise one or more targets selected from a Naïve Bayes (NB) classifier. The plurality of targets may comprise 2, 3, 4, 5, 6, 7, 8, 9, 10 or more targets selected from an NB classifier. The plurality of targets may comprise 12, 13, 14, 15, 17, 20, 22, 25 or more targets selected from an NB classifier. For example, a NB classifier of the present invention may comprise two or more targets selected from Table 2.

The plurality of targets may comprise one or more targets selected from a recursive partitioning (Rpart) classifier. The plurality of targets may comprise 2, 3, 4, 5, 6, 7, 8, 9, 10 or more targets selected from an Rpart classifier. The plurality of targets may comprise 12, 13, 14, 15, 17, 20, 22, 25 or more targets selected from an Rpart classifier. For example, an Rpart classifier of the present invention may comprise two or more targets selected from Table 2.

The plurality of targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10, 11, 12, 13, 14, 15, 17, 20, 22, 25 or more targets selected from a high dimensional discriminate analysis (HDDA) classifier. For example, an Rpart classifier of the present invention may comprise two or more targets selected from Table 2.

### Probes/Primers

The present invention provides for a probe set for diagnosing, monitoring and/or predicting a status or outcome of breast cancer in a subject comprising a plurality of probes, wherein (i) the probes in the set are capable of detecting an expression level of at least one target; and (ii) the expression level determines the cancer status (e.g., risk of recurrence) of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from breast cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly Such modified or substituted polynucleotide probes may provide desirable properties such as, for example, enhanced affinity for a target gene and increased stability. The probe set may comprise a coding target and/or a non-coding target.

In some embodiments, the probe set comprise a plurality of target sequences that hybridize to at least about 5 coding targets and/or non-coding targets. Alternatively, the probe set comprise a plurality of target sequences that hybridize to at least about 10 coding targets and/or non-coding targets. In some embodiments, the probe set comprise a plurality of target sequences that hybridize to at least about 15 coding targets and/or non-coding targets. In some embodiments, the probe set comprise a plurality of target sequences that hybridize to at least about 20 coding targets and/or non-coding targets. In some embodiments, the probe set comprise a plurality of target sequences that hybridize to at least about 30 coding targets and/or non-coding targets.

The system of the present invention further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the invention, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

In one embodiment, the primers or primer pairs, when used in an amplification reaction, specifically amplify at least a portion of a nucleic acid sequence of a target (or subgroups thereof as set forth herein), an RNA form thereof, or a complement to either thereof.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In some embodiments, polynucleotides of the invention comprise at least 20 consecutive bases of the nucleic acid sequence of a target or a complement thereto. The polynucleotides may comprise at least 21, 22, 23, 24, 25, 27, 30, 32, 35, 40, 45, 50, or more consecutive bases of the nucleic acids sequence of a target.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### Diagnostic Samples

A biological sample containing breast cancer cells is collected from a subject in need of treatment for cancer to evaluate if the subject is at low risk of cancer recurrence based on an expression level or expression profile and likely to benefit from adjuvant radiotherapy. Diagnostic samples for use with the systems and in the methods of the present invention comprise nucleic acids suitable for providing RNA expression information. In principle, the biological sample from which the expressed RNA is obtained and analyzed for target gene expression can be any material suspected of comprising cancerous breast tissue or cells. The diagnostic sample can be a biological sample used directly in a method of the invention. Alternatively, the diagnostic sample can be a sample prepared from a biological sample.

In one embodiment, the sample or portion of the sample comprising or suspected of comprising cancerous tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from a breast tumor biopsy.

The samples may be archival samples, having a known and documented medical outcome, or may be samples from current subjects whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P, Paramat, Paramat Extra, Paraplast, Paraplast X-tra, Paraplast Plus, Peel Away Paraffin Embedding Wax, Polyester Wax, Carbowax Polyethylene Glycol, Polyfin, Tissue Freezing Medium TFMFM, Cryo-Gef, and OCT Compound (Electron Microscopy Sciences, Hatfield, Pa.). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly Mass., High Pure FFPE RNA Micro Kit, Roche Applied Science, Indianapolis, Ind.). RNA can be extracted from frozen tissue sections using TRIzol (Invitrogen, Carlsbad, Calif.) and purified using RNeasy Protect kit (Qiagen, Valencia, Calif.). RNA can be further purified using DNAse I treatment (Ambion, Austin, Tex.) to eliminate any contaminating DNA. RNA concentrations can be made using a Nanodrop ND-1000 spectrophotometer (Nanodrop Technologies, Rockland, Del.). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers are suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100, 110, or more primers or pairs of primers suitable for selectively amplifying one or more targets.

In some embodiments, the primers or primer pairs of the kit, when used in an amplification reaction, specifically amplify a non-coding target, coding target, exonic, or non-exonic target described herein, a nucleic acid sequence corresponding to a target selected from Table 2, an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target selected from Table 2, RNA forms thereof, or complements thereto. At least two, three, four or five primers or pairs of primers suitable for selectively amplifying the one or more targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from subjects showing no evidence of disease, as well as tissue and/or nucleic acids obtained from or representative of tumor samples from subjects that develop systemic cancer.

The nucleic acids may be provided in an array format, and thus an array or microarray may be included in the kit. The kit optionally may be certified by a government agency for use in prognosing the disease outcome of cancer subjects and/or for designating a treatment modality.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target genes.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tf7, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, Calif.), including the WT-Ovation™ System, WT-Ovation™ System v2, WT-Ovation™ Pico System, WT-Ovation™ FFPE Exon Module, WT-Ovation™ FFPE Exon Module RiboAmp and RiboAmp^{Plus} RNA Amplification Kits (MDS Analytical Technologies (formerly Arcturus) (Mountain View, Calif.), Genisphere, Inc. (Hatfield, Pa.), including the RampUp Plus and SenseAmp RNA Amplification kits, alone or in combination. Amplified nucleic acids may be subjected to one or more purification reactions after amplification and labeling, for example using magnetic beads (e.g., RNAC lean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert® Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.).

Detection and/or Quantification of Target Genes

Any method of detecting and/or quantitating the expression of the encoded target genes can in principle be used in the invention. The expressed target genes can be directly detected and/or quantitated, or may be copied and/or amplified to allow detection of amplified copies of the expressed target genes.

Methods for detecting and/or quantifying a target gene can include Northern blotting, sequencing, array or microarray hybridization, by enzymatic cleavage of specific structures (e.g., a Clariom S assay, ThermoFisher Scientific, an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis. Methods for detecting and/or quantifying a target gene can include gene-level expression analysis of annotated genes using microarray hybridization (e.g., GeneChip Human Exon 1.0 ST assay or Clariom S assay, ThermoFisher Scientific).

In some instances, target genes may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target gene include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for ORT-PCR Analysis

Reverse transcription can be performed by any method known in the art. For example, reverse transcription may be performed using the Omniscript kit (Qiagen, Valencia, Calif.), Superscript III kit (Invitrogen, Carlsbad, Calif.), for RT-PCR. Target-specific priming can be performed in order to increase the sensitivity of detection of target genes and generate target-specific cDNA.

### TaqMan® Gene Expression Analysis

TaqMan®RT-PCR can be performed using Applied Biosystems Prism (ABI) 7900 HT instruments in a 5 1.11 volume with target gene-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present invention contemplates that a probe set or probes derived therefrom may be provided in an array format. In the context of the present invention, an “array” is a spatially or logically organized collection of polynucleotide probes. An array comprising probes specific for a coding target, non-coding target, or a combination thereof may be used. Alternatively, an array comprising probes specific for two or more of transcripts of a target, or a product derived thereof, can be used. Desirably, an array may be specific for 5, 10, 15, 20, 25, 30 or more of transcripts of a target gene. Expression of these genes may be detected alone or in combination with other transcripts. In some embodiments, an array is used which comprises a wide range of sensor probes for breast-specific expression products, along with appropriate control sequences. In some instances, the array may comprise the Human Exon 1.0 ST Array (HuEx 1.0 ST, Thermo Fisher Scientific, Santa Clara, Calif.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present invention, the Cancer Prognosticarray is a chip.

Data Analysis

In some embodiments, one or more pattern recognition methods can be used in analyzing the expression level of target genes. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a subject who may exhibit no evidence of disease, who may exhibit systemic cancer, or who may exhibit biochemical recurrence or locoregional recurrence. The likelihood score can be used to distinguish these disease states. The models and/or algorithms can be provided in machine readable format, and may be used to correlate expression levels or an expression profile with a disease state, and/or to designate a treatment modality for a subject or class of subjects.

Assaying the expression level for a plurality of target genes may comprise the use of an algorithm or classifier. Array data can be managed, classified, and analyzed using techniques known in the art. Assaying the expression level for a plurality of gene targets may comprise probe set modeling and data pre-processing. Probe set modeling and data pre-processing can be derived using the Robust Multi-Array (RMA) algorithm or variants GC-RMA, fRMA, Probe Logarithmic Intensity Error (PLIER) algorithm or variant iterPLIER. Variance or intensity filters can be applied to pre-process data using the RMA algorithm, for example by removing target genes with a standard deviation of <10 or a mean intensity of <100 intensity units of a normalized data range, respectively.

Alternatively, assaying the expression level for a plurality of gene targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine, Lasso and Elastic-Net Regularized General Linear models), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Subtyping

Molecular subtyping is a method of classifying breast cancer into one of multiple genetically-distinct categories, or subtypes. Each subtype responds differently to different kinds of treatments, and the presence of a particular subtype is predictive of, for example, radioresistance or chemoresistance, higher or lower risk of recurrence, or good or poor prognosis for an individual. Differential expression analysis of a plurality of the gene targets listed in Table 2 allows for the identification of subjects at low risk of cancer recurrence who, for example, may benefit most from adjuvant radiotherapy. In some instances, the molecular subtyping methods of the present invention are used in combination with other biomarkers, like tumor grade and hormone levels, for analyzing the breast cancer. For example, a subject with estrogen receptor positive (ER+), human epidermal growth factor receptor 2 negative (HER2-) breast cancer, node-negative breast cancer, who is post-menopausal, may be more likely to have a lower risk of recurrence (e.g., locoregional recurrence).

### Cancer Recurrence

Cancer recurrence is the return of cancer after a period when no cancer cells are detected in the body. Following surgery for operable breast cancer, disease can recur locally, regionally, and/or at distant metastatic sites. A local recurrence is the reappearance of cancer on the ipsilateral chest wall. In contrast, a regional recurrence denotes tumor involving the regional lymph nodes, usually ipsilateral axillary or supraclavicular, and less commonly infraclavicular and/or internal mammary nodes. Some breast cancer patients will have local or locoregional recurrence after breast-conserving surgery and radiotherapy within ten years of first being diagnosed with breast cancer. If the breast was removed in the course of initial treatment, these women will have a local recurrence in the armpit or the chest wall within ten years. In some embodiments, the subject treated in the methods of the present invention has a node-negative breast cancer.

### Therapeutic Regimens

Diagnosing, predicting, or monitoring a status or outcome of a cancer may comprise treating a cancer or preventing a cancer progression. In addition, diagnosing, predicting, or monitoring a status or outcome of a cancer may comprise identifying or predicting that a subject is at low or high risk of a recurrence (e.g., locoregional recurrence). In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. For example, a subject determined to be at low risk of recurrence of breast cancer based on expression profiling, as described herein, may be spared adjuvant radiotherapy. In some instances, a subject determined to be at high risk of recurrence of breast cancer based on expression profiling, as described herein, may be treated with mastectomy, radiation boost, or adjuvant systemic therapy. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include hormonal/endocrine therapy, surgery, chemotherapy, radiation therapy, immunotherapy/biological therapy, and photodynamic therapy.

Hormonal therapy or endocrine therapy may involve administration of hormones, such as steroid hormones or hormone antagonists to modulate the levels of certain hormones in order to arrest growth or induce apoptosis of hormone-responsive cancer cells. For example, breast cancer may be treated with a selective estrogen receptor modulator (SERM) such as, but not limited to, tamoxifen, raloxifene, and toremifene. Alternately or additionally, inhibitors of hormone synthesis such as aromatase inhibitors, including, but not limited to, letrozole, anastrozole, exemestane, and aminoglutethimide may be used to treat breast cancer. In some cases, hormone supplementation with progestins such as, but not limited to, megestrol acetate and medroxyprogesterone acetate may be used for the treatment of hormone-responsive, advanced breast cancer. In particular, ER+ breast cancer can be treated with either an estrogen receptor antagonist (e.g. tamoxifen) or a drug that blocks the production of estrogen such as an aromatase inhibitor (e.g. anastrozole or letrozole). Hormonal therapy may also include surgical removal of endocrine organs (e.g., orchiectomy or oophorectomy).

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antiobiotics include, but are not limited to, actinomycin, anthracyclines, doxorubicin, daunorubicin, valrubicin, idarubicin, epirubicin, bleomycin, plicamycin, and mitomycin.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning) In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the subject or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the subject in the same manner as a normal CT scanner. Tomotherapy machines can capture CT images of the subject's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and subject positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, subjects may receive radiation therapy to the entire brain (called whole-brain radiation therapy) in addition to SRS. SRS requires the use of a head frame or other device to immobilize the subject during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a breast tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in subjects using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat subjects with breast cancer who have undergone breast-conserving surgery.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the subject. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a subject may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to subjects first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Biological therapy (sometimes called immunotherapy, biotherapy, biologic therapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit subjects undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a subject's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, prostate and breast.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in subjects with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

Cancer vaccines are another form of biological therapy. Cancer vaccines may be designed to encourage the subject's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a subject's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outsubject procedure. PDT may also be repeated and may be used with other therapies, such as surgery, radiation, or chemotherapy.

Extracorporeal photopheresis (ECP) is a type of PDT in which a machine may be used to collect the subject's blood cells. The subject's blood cells may be treated outside the body with a photosensitizing agent, exposed to light, and then returned to the subject. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in subjects for whom the usual treatments are not appropriate, and to relieve symptoms in subjects with non-small cell lung cancer that obstructs the airways. Porfimer sodium may also be used for the treatment of precancerous lesions in subjects with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a subject's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO₂) lasers, argon lasers, and neodymium:yttrium-aluminum-garnet (Nd:YAG) lasers. Each of these can shrink or destroy tumors and can be used with endoscopes. CO₂ and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

For subjects with systemic disease, additional treatment modalities such as adjuvant chemotherapy (e.g., docetaxel, mitoxantrone and prednisone) and systemic radiation therapy (e.g., samarium or strontium) can be designated. Such subjects would likely be treated immediately with radiation therapy in order to eliminate presumed micro-metastatic disease, which cannot be detected clinically but can be revealed by the target gene expression signature. Such subjects can also be more closely monitored for signs of disease progression.

For subjects that do not have systemic disease, localized adjuvant radiation therapy (e.g., localized to breast tissue) or endocrine therapy or chemotherapy may be administered. For subjects with no evidence of disease (NED), expression profiling and/or calculation of a risk score, as described herein, may be used to determine the risk of recurrence of the breast cancer. For subjects identified as having a low risk of recurrence of breast cancer and identified as having a high benefit of radiotherapy using the methods described herein, adjuvant radiation therapy would be recommended.

Target genes can be grouped so that information obtained about the set of target genes in the group can be used to make or assist in making a clinically relevant judgment such as a diagnosis, prognosis, or treatment choice.

A subject report is also provided comprising a representation of measured expression levels of a plurality of target genes in a biological sample from the subject, wherein the representation comprises expression levels of target genes corresponding to any one, two, three, four, five, six, eight, ten, twenty, thirty or more of the target genes, the subsets described herein, or a combination thereof. In some embodiments, the representation of the measured expression level(s) may take the form of a linear or nonlinear combination of expression levels of the target genes of interest. The subject report may be provided in a machine (e.g., a computer) readable format and/or in a hard (paper) copy. The report can also include standard measurements of expression levels of said plurality of target genes from one or more sets of subjects with known disease status and/or outcome. The report can be used to inform the subject and/or treating physician of the expression levels of the expressed target genes, the likely medical diagnosis and/or implications, and optionally may recommend a treatment modality for the subject.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above. The articles may also have gene expression profiles digitally recorded therein so that they may be compared with gene expression data from subject samples. Alternatively, the profiles can be recorded in different representational format. A graphical recordation is one such format. Clustering algorithms can assist in the visualization of such data.

### EXAMPLES

### Example 1: Development and Validation of a Genomic Classifier for Prognosis of Local

Recurrence of Breast Cancer and Prediction of Response to Radiation Therapy.

A genomic classifier for the prognosis of local recurrence of breast cancer and prediction of response to radiation therapy was developed as follows. In this study, previous genomic classifiers were evaluated for local recurrence (LRR) and radiotherapy (RT) response in the SweBCG 91-RT trial, as well as validate a novel signature derived from publicly available datasets.

### Validation Dataset

The SweBCG 91-RT trial was a multi-institutional trial randomizing women with stage I-II lymph node-negative breast cancer to postoperative whole breast RT or no RT after BCS. (Sjostrom M et al. Journal of clinical oncology: official journal of the American Society of Clinical Oncology 2017; 35(28): 3222-9 and Malmström P et al. European journal of cancer (Oxford, England: 1990) 2003; 39(12): 1690-7.) In the trial, all subsets of patients benefitted from radiation therapy and no clinical subgroup analyzed in the trial could be spared radiation, including a low-risk group of patients above 64 years of age with hormone receptor positive tumors smaller than 21 mm. (Killander F et al. European journal of cancer (Oxford, England: 1990) 2016; 67: 57-65.) The SweBCG 91-RT trial randomized stage I-IIA node-negative breast cancer patients to postoperative whole breast RT or to no RT following BCS. The original study found a benefit of radiotherapy for the local and regional recurrence endpoints, but not to metastasis or breast cancer death. Out of the original 1,178 patients, primary tumor tissue derived from formalin-fixed paraffin-embedded (FFPE) samples from 765 patients were successfully profiled on the GeneChip Human Exon 1.0 ST array (ThermoFisher). Complete locoregional recurrence information was available for further analysis in 764 patients. Data is available at Gene Expression Omnibus with accession number GSE119295. The majority (92%) of patients were not treated with adjuvant systemic therapy. Clinical details can be found in Table 1 below. The median follow-up was 14.0 years for LRR in patients free from event.

TABLE 1

| | All Patients | | No RT | | RT | |
|---|---|---|---|---|---|---|
| Total number of patients | 748 | | 392 | | 356 | |
| Age at Surgery (years), median (range) | 59 | (31-78) | 59 | (33-78) | 59 | (31-78) |
| Post-menopausal | | | | | | |
| No | 149 | (20%) | 86 | (23%)) | 63 | (18%) |
| Yes | 578 | (80%) | 293 | (77%) | 285 | (82%) |
| Missing | 21 | | 13 | | 8 | |
| Histological grade | | | | | | |
| 1 | 98 | (13%) | 45 | (12%) | 53 | (15%) |
| 2 | 450 | (61%) | 236 | (61%) | 214 | (61%) |
| 3 | 188 | (26%) | 107 | (28%) | 81 | (23%) |
| missing | 12 | | 4 | | 8 | |
| Tumor Size (mm), median (range) | 12 | (1-40) | 12 | (1-40) | 12 | (2-30) |
| Estrogen receptor status | | | | | | |
| Negative | 88 | (12%) | 50 | (13%) | 38 | (11%) |
| Positive | 656 | (88%) | 341 | (87%) | 315 | (89%) |
| Missing | 4 | | 1 | | 3 | |
| Progesterone receptor status | | | | | | |
| Negative | 201 | (27%) | 102 | (26%) | 99 | (28%) |
| Positive | 543 | (73%) | 289 | (74%) | 254 | (72%) |
| Missing | 4 | | 1 | | 3 | |
| HER2 status | | | | | | |
| Negative | 685 | (93%) | 363 | (94%) | 322 | (92%) |
| Positive | 54 | (7%) | 25 | (6%) | 29 | (8%) |
| Missing | 9 | | 4 | | 5 | |
| Subtype by IHC | | | | | | |
| HER2+ | 54 | (7%) | 25 | (6%) | 29 | (8%) |
| Luminal A | 410 | (55%) | 217 | (56%) | 193 | (55%) |
| Luminal B (HER2−) | 210 | (28%) | 107 | (28%) | 103 | (29%) |
| Triple negative | 65 | (9%) | 39 | (0%) | 26 | (7%) |
| Missing | 9 | | 4 | | 5 | |
| Adjuvant Endocrine therapy | | | | | | |
| No | 694 | (93%) | 359 | (92%) | 335 | (94%) |
| Yes | 54 | (7%) | 33 | (8%) | 21 | (6%) |
| Missing | 0 | | 0 | | 0 | |
| Adjuvant Chemotherapy | | | | | | |
| No | 738 | (99%) | 386 | (99%) | 352 | (99%) |
| Yes | 10 | (1%) | 6 | (2%) | 4 | (1%) |
| Missing | 0 | | 0 | | 0 | |
| Ipsilateral breast tumor recurrence | 149 | (20%) | 103 | (26%) | 46 | (13%) |
| Regional recurrence | 32 | (4%) | 22 | (6%) | 10 | (3%) |
| Distant recurrence | 106 | (14%) | 61 | (16%) | 45 | (13%) |
| Died from breast cancer | 136 | (18%) | 75 | (19%) | 61 | (17%) |
| Died from any cause | 355 | (48%) | 191 | (49%) | 164 | (46%) |

### Computation of Previously-Published Breast Cancer Risk Scores

Eight previously-published genomic signatures developed for radiation sensitivity or local and loco-regional recurrence were examined. The Cui 2018 signature is a 34-gene signature with genes selected from radiation-related gene sets and associated with local recurrence-free survival in a cohort of early stage breast cancer. (Cui Y et al. Clinical cancer research: an official journal of the American Association for Cancer Research 2018.) The Eschrich 2009 signature is a 10-gene rank-based linear model including genes identified for prediction of radiosensitivity across cell lines for multiple human cancer types. (Eschrich S A et al. International journal of radiation oncology, biology, physics 2009; 75(2): 489-96.) The Speers 2015 signature is a 51-gene random forest model comprised of genes predictive of radiosensitivity in breast cancer cell lines and trained for local recurrence in a cohort of early stage breast cancer. (Speers C et al. Clinical cancer research: an official journal of the American Association for Cancer Research 2015; 21(16): 3667-77.) Top-scoring pairs (TSP) signatures, the TSP intensification and omission signatures, are estrogen receptor (ER) stratified signatures with around 200 genes selected both from literature and from training in a cohort of early stage breast cancer. (Sjostrom M et al. Breast cancer research: BCR 2018; 20(1): 64.) The Zhang 2016 signature is comprised of 14 genes related to genomic instability, prognostic for overall survival in three breast cancer datasets. (Zhang W et al. Nature communications 2016; 7: 12619.) The 21-gene and 70-gene signatures were both developed for the metastasis endpoint, but have been evaluated for local-regional recurrence in secondary analyses of trials non-randomized to RT. (Mamounas E P et al. Journal of the National Cancer Institute 2017; 109(4); Mamounas E P et al. Journal of clinical oncology: official journal of the American Society of Clinical Oncology 2010; 28(10): 1677-83; and Drukker C A et al. Breast cancer research and treatment 2014; 148(3): 599-613.) These previously-published breast cancer risk scores were developed on a variety of platforms. We applied gene expression data from the GeneChip Human Exon 1.0 ST microarrays to genomic signature equations to calculate surrogate continuous risk scores using the equations as defined in the original publications. See Supplemental Methods for details on score calculation. When examining high vs. low risk scores, we applied a cut off at the 75^{th} percentile for all signature scores. This is done to allow a direct comparison between signatures before determining a final clinical cut-off, and reflects the rate of locoregional recurrence (32% in the SweBCG 91-RT no RT arm), and this approached has been used previously. (Eschrich S A et al. Clinical cancer research: an official journal of the American Association for Cancer Research 2012; 18(18): 5134-43.)

### Training Datasets for Novel Classifier

To train and select the novel classifier, we used three publicly available early stage breast cancer gene expression datasets with detailed local recurrence information. These expression datasets were the Servant dataset, (Servant N et al. Clinical cancer research: an official journal of the American Association for Cancer Research 2012; 18(6): 1704-15) the van de Vijver dataset, (van de Vijver M J et al. The New England journal of medicine 2002; 347(25): 1999-2009) and the Lund fresh frozen dataset. (Sjostrom M et al. Breast cancer research: BCR 2018; 20(1): 64.) We ensured that patients were not duplicated among these cohorts and the SweBCG 91-RT trial.

Development of a New Local Recurrence Classifier that is Suitable for FFPE Tissue

In order to derive a new local recurrence signature that could work in both fresh frozen and FFPE based cohorts, information was used from 16 patients in common between the Lund Fresh Frozen and the SweBCG 91-RT datasets in order to select genes with a large dynamic range of expression in both tissue types and to select genes with high correlation of expression between tissue types. For these 16 patients and for all genes in common between the four datasets, genes were ranked by their correlation with local recurrence with FDR adjusted p-value for Spearman's correlation coefficient. Genes were also ranked by their variance of expression for the 16 fresh frozen and 16 FFPE samples, separately.

The Servant cohort, as it was the largest publicly available cohort with 343 samples, was used for feature selection and model training Within the Servant cohort, age was the most prognostic clinical variable to the local recurrence endpoint (FDR<0.05). For gene selection and model selection, feature selection parameters were varied as well as model parameters, and a model was selected that minimized the product of the p-values in a Cox proportional hazards model (Coxph) in the van de Vijver and Lund FF datasets. Among genes with a variance of expression greater than the median expression, as calculated within the 16 paired fresh frozen and FFPE samples, genes with a Coxph FDR<0.1 to the local recurrence endpoint were included, resulting in 27 genes selected for the model. As age was the strongest clinical factor for the endpoint in the training dataset, it was included in the model, resulting in a final clinical-genomic classifier of 27 genes and age (See Table 2). The classifier was locked before external validation in the SweBCG91RT trial

TABLE 2
The 27 Genes in the exemplary Genomic Classifier

| | Entrez | | | |
|---|---|---|---|---|
| Variable | Gene ID | Gene Name | Biological Function | Coefficient |
| ABCC8 | 6833 | ATP binding cassette | potassium ion transport | −0.041 |
| | | subfamily C member 8 | | |
| BTG3 | 10950 | BTG anti-proliferation factor 3 | cell proliferation, cell cycle | 0.102 |
| CCNB1 | 891 | cyclin B1 | G2/M transition of mitotic | 0.015 |
| | | | cell cycle | |
| CENPF | 1063 | centromere protein F | cell cycle | −0.089 |
| CKMT1B | 1159 | creatine kinase, mitochondrial 1B | transferase activity | 0.164 |
| CNIH4 | 29097 | cornichon family AMPA receptor | protein transport | 0.097 |
| | | auxiliary protein 4 | | |
| CRABP2 | 1382 | cellular retinoic acid binding | transcription, signal transduction | 0.14 |
| | | protein 2 | | |
| ECHDC2 | 55268 | enoyl-CoA hydratase domain | fatty acid beta-oxidation | 0.02 |
| | | containing 2 | | |
| EEF2K | 29904 | eukaryotic elongation factor | calcium ion binding, protein | −0.195 |
| | | 2 kinase | kinase activity | |
| EIF3L | 51386 | eukaryotic translation initiation | translational initiation | −0.258 |
| | | factor 3 subunit L | | |
| ENTPD6 | 955 | ectonucleoside triphosphate | response to magnesium ion, | −0.655 |
| | | diphosphohydrolase 6 (putative) | response to calcium ion | |
| EPHX2 | 2053 | epoxide hydrolase 2 | phosphatase activity | −0.155 |
| H2AFZ | 3015 | H2A histone family member Z | transcription regulation | 0.044 |
| HSD17B4 | 3295 | hydroxysteroid 17-beta | 3-hydroxyacyl-CoA dehydrogenase | −0.154 |
| | | dehydrogenase 4 | activity | |
| KPNA2 | 3838 | karyopherin subunit alpha 2 | regulation of DNA recombination, | 0.009 |
| | | | DNA metabolic process | |
| LEF1 | 51176 | lymphoid enhancer binding | transcription regulation, EMT | −0.105 |
| | | factor 1 | | |
| N4BP2L1 | 90634 | NEDD4 binding protein 2 like 1 | kinase activity | −0.697 |
| NEK10 | 152110 | NIMA related kinase 10 | kinase activity | 0.169 |
| PFDN4 | 5203 | prefoldin subunit 4 | protein folding | 0.27 |
| PSD3 | 23362 | pleckstrin and Sec7 domain | phospholipid binding | −0.106 |
| | | containing 3 | | |
| RUNX1 | 861 | runt related transcription | transcription regulation | 0.046 |
| | | factor 1 | | |
| SEC14L2 | 23541 | SEC14 like lipid binding 2 | transcription, DNA-templated | 0.04 |
| STK39 | 27347 | serine/threonine kinase 39 | protein kinase activity | −0.112 |
| TBC1D8 | 11138 | TBC1 domain family member 8 | GRPase activator activity | −0.297 |
| TMSB15A | 11013 | thymosin beta 15a | regulation of cell migration | 0.03 |
| USO1 | 8615 | USO1 vesicle transport factor | ER to Golgi vesicle-mediated | 0.402 |
| | | | transport | |
| ZBTB16 | 7704 | zinc finger and BTB domain | transcription regulation | −0.013 |
| | | containing 16 | | |
| age | NA | | | −0.026 |

### Data Analysis

The author responsible for deriving the previously-published signature equations and for deriving the new classifier was blinded from the SweBCG 91-RT cohort. The classifier was validated using Cox regression with LRR as the primary endpoint, and hazard ratios (HRs) are calculated when using the top 25^{th} percentile as high scores, except for testing for interaction where the continuous score was used.

### Results

### Performance of Previously-Published Signatures of Local Recurrence and Radiosensitivity in the SweBG91-RT Cohort

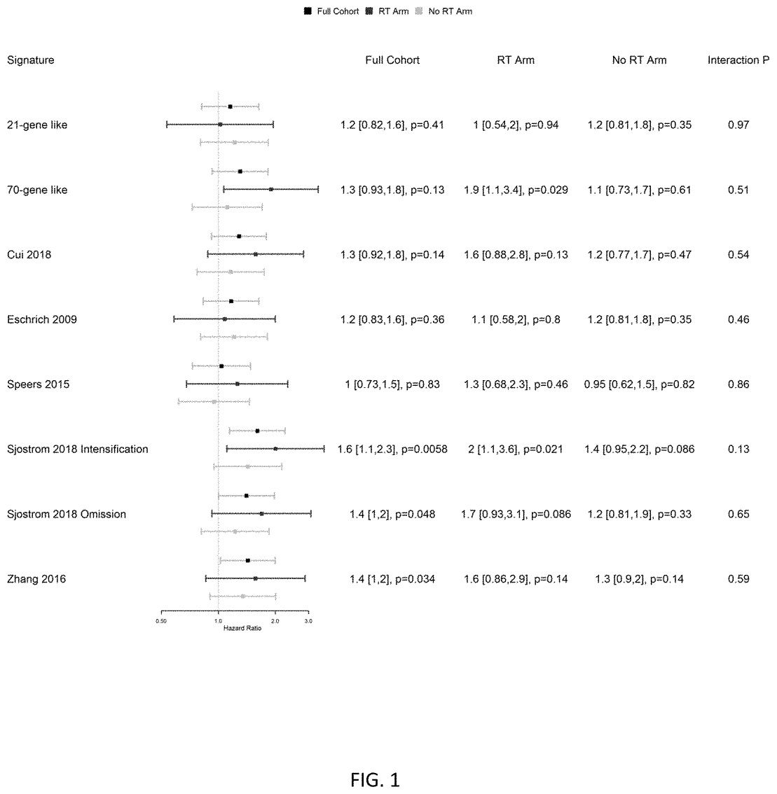

Eight previously-published signatures specific for radiation sensitivity were examined for their ability to prognosticate for local recurrence and predict for benefit of radiation therapy in 748 patients of the SweBCG91RT cohort, 356 of whom were treated with RT, and 392 of whom were not (FIG. 1). Three of the eight signatures, the TSP intensification, the TSP omission, and the Zhang 2016 signatures, were prognostic in the full cohort (P<0.05) for the locoregional recurrence endpoint in univariate Cox analysis. The signatures had a higher HR in the RT arm, compared to overall or the no RT arm (FIG. 1). Two of eight signatures were still prognostic (P<0.05) in multivariate analysis, including chemotherapy, hormone therapy, subtype, and grade (Table 3). However, none of the previously-published signatures had a significant interaction with radiation (p<0.05), and the effect of RT is consistent for high and low scores for all eight signatures (FIG. 1), as demonstrated by the similar reduction in LRR rates for the no RT and RT arms regardless of high or low score.

TABLE 3
Previous signatures cox modelling

| | All HR | | All P | RT HR | | RT P | No RT HR | | No RT P |
|---|---|---|---|---|---|---|---|---|---|
| Non-adjusted | | | | | | | | | |
| Eschrich 2009 | 1.2 | [0.83, 1.6] | 0.36 | 1.1 | [0.58, 2] | 0.8 | 1.2 | [0.81, 1.8] | 0.35 |
| Speers 2015 | 1 | [0.73, 1.5] | 0.83 | 1.3 | [0.68, 2.3] | 0.46 | 0.95 | [0.62, 1.5] | 0.82 |
| Zhang 2016 | 1.4 | [1, 2] | 0.034 | 1.6 | [0.86, 2.9] | 0.14 | 1.3 | [0.9, 2] | 0.14 |
| Sjostrom 2018 Intensification | 1.6 | [1.1, 2.3] | 0.0058 | 2 | [1.1, 3.6] | 0.021 | 1.4 | [0.95, 2.2] | 0.086 |
| Sjostrom 2018 Omission | 1.4 | [1, 2] | 0.048 | 1.7 | [0.93, 3.1] | 0.086 | 1.2 | [0.81, 1.9] | 0.33 |
| Cui 2018 | 1.3 | [0.92, 1.8] | 0.14 | 1.6 | [0.88, 2.8] | 0.13 | 1.2 | [0.77, 1.7] | 0.47 |
| 21-gene-like | 1.2 | [0.82, 1.6] | 0.41 | 1 | [0.54, 2] | 0.94 | 1.2 | [0.81, 1.8] | 0.35 |
| 70-gene-like | 1.3 | [0.93, 1.8] | 0.13 | 1.9 | [1.1, 3.4] | 0.029 | 1.1 | [0.73, 1.7] | 0.61 |
| MVA adjusted for grade, | | | | | | | | | |
| sbt and systemic treatment | | | | | | | | | |
| Eschrich 2009 | 1.23 | [0.871, 1.73] | 0.243 | 1.15 | [0.614, 2.15] | 0.666 | 1.29 | [0.854, 1.95] | 0.226 |
| Speers 2015 | 0.889 | [0.608, 1.3] | 0.546 | 1.24 | [0.637, 2.42] | 0.525 | 0.776 | [0.487, 1.24] | 0.287 |
| Zhang 2016 | 1.37 | [0.895, 2.09] | 0.148 | 2.31 | [1.04, 5.12] | 0.0391 | 1.13 | [0.692, 1.85] | 0.625 |
| Sjostrom 2018 Intensification | 1.78 | [1.15, 2.74] | 0.0093 | 2.61 | [1.27, 5.35] | 0.00888 | 1.43 | [0.823, 2.5] | 0.203 |
| Sjostrom 2018 Omission | 1.55 | [1.03, 2.32] | 0.0343 | 2.21 | [1.1, 4.42] | 0.0259 | 1.3 | [0.784, 2.14] | 0.312 |
| Cui 2018 | 1.21 | [0.857, 1.7] | 0.282 | 1.46 | [0.802, 2.67] | 0.215 | 1.09 | [0.719, 1.67] | 0.673 |
| 21-gene-like | 1.06 | [0.657, 1.71] | 0.808 | 0.929 | [0.377, 2.29] | 0.874 | 1.1 | [0.624, 1.95] | 0.738 |
| 70-gene-like | 1.26 | [0.816, 1.96] | 0.294 | 2.55 | [1.23, 5.32] | 0.0122 | 1.02 | [0.594, 1.77] | 0.93 |

### Novel Genomic Classifier Independently Validates as Prognostic for Loco-Regional Recurrence and Predictive for Radiation Therapy

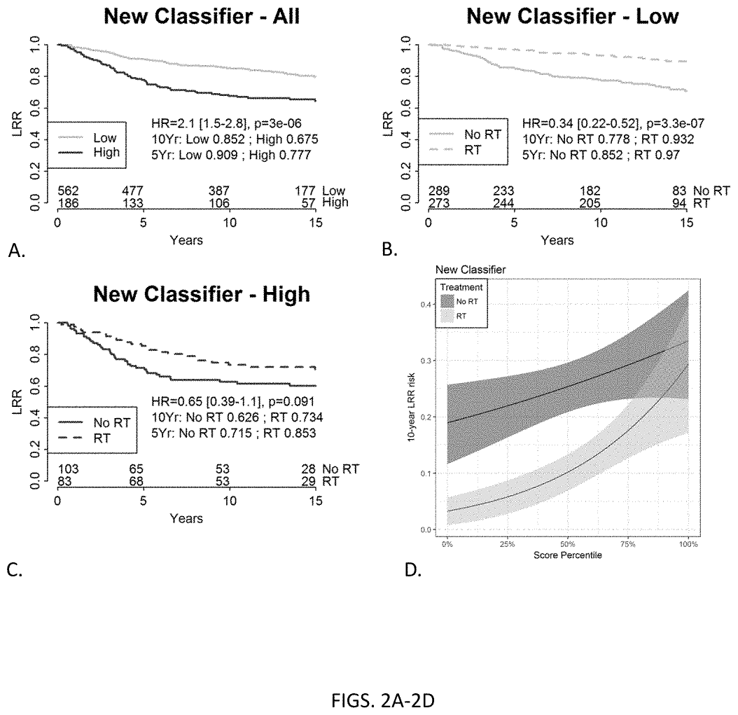

Given that previously-published signatures in breast cancer were not strongly prognostic for LRR nor predictive for benefit of radiation therapy, we derived a new gene expression-based signature in publicly available cohorts with gene expression data from fresh frozen tissue samples, and then independently validated the novel signature in SweBCG 91-RT. The genomic-clinical classifier was derived, trained and locked in public data before being sent for validation in the SweBCG91RT cohort. The score was assessed using the same cutoff as for the previously-published signatures, where patients with the top 25^{th} percentile of scores are considered at high risk for local recurrence. The signature was prognostic for LRR in the full cohort (HR=2.1[1.5-2.8], p<0.001) (FIG. 2A). Patients with low scores and at low risk for LRR have a high benefit from RT, where those treated with RT have a third of the risk of LRR (HR=0.34[0.22-0.52], p<0.001 for RT compared to No RT arms), and at 10 years, there is 15% reduction in risk of LRR for patients who received RT compared to those who did not (10 year LRR rate for patients with low classifier scores who did not receive RT=0.22, for those who did receive RT=0.07) (FIG. 2B). However, patients with high scores do not have a significant benefit from RT (HR=0.65[0.39-1.1], p=0.091, 10-year LRR rate for patients with low classifier scores who did not receive RT=0.37, for those who did receive RT=0.27) (FIG. 2C). The classifier is predictive of RT benefit and has a significant interaction with RT (p_{interaction}=0.008), which remains statistically significant when including systemic therapy, subtype, and grade in multivariable analysis (p=0.009) (FIG. 2D and Table 4 and Table 5).

TABLE 4
Exemplary genomic classifier multivariate analysis of prognostic potential.

| | All - UVA | | | All - MVA | | | RT - UVA | | |
|---|---|---|---|---|---|---|---|---|---|
| | HR | | P | HR | | P | HR | | P |
| New Classifier | 2.08 | [1.52, 2.84] | 4.94E−06 | 2.01 | [1.46, 2.78] | 2.01E−05 | 3.14 | [1.81, 5.45] | 4.54E−05 |
| Subtype by IHC | Luminal A as Reference | | | | | | | | |
| Luminal B (HER2−) | 1.07 | [0.752, 1.52] | 0.71 | 0.978 | [0.678, 1.41] | 0.91 | 0.64 | [0.323, 1.27] | 0.20 |
| Triple Negative | 1.21 | [0.686, 2.12] | 0.52 | 0.979 | [0.517, 1.85] | 0.95 | 0.824 | [0.252, 2.69] | 0.75 |
| HER2+ | 1.31 | [0.746, 2.3] | 0.35 | 1.14 | [0.619, 2.1] | 0.67 | 1.15 | [0.447, 2.95] | 0.77 |
| Grade | Grade 1 as Reference | | | | | | | | |
| Grade 2 | 1.93 | [1.08, 3.43] | 0.03 | 1.88 | [1.05, 3.36] | 0.03 | 1.81 | [0.704, 4.64] | 0.22 |
| Grade 3 | 2.48 | [1.35, 4.58] | 3.61E−03 | 2.21 | [1.14, 4.29] | 0.02 | 2.3 | [0.818, 6.47] | 0.11 |
| Systemic Therapy | 0.461 | [0.216, 0.983] | 0.04 | 0.414 | [0.191, 0.898] | 0.03 | 0.525 | [0.128, 2.16] | 0.37 |

| | RT - MVA | | | No RT - UVA | | | No RT - MVA | | |
|---|---|---|---|---|---|---|---|---|---|
| | HR | | P | HR | | P | HR | | P |
| New Classifier | 3.14 | [1.78, 5.54] | 7.52E−05 | 1.67 | [1.14, 2.46] | 0.01 | 1.61 | [1.08, 2.39] | 0.02 |
| Subtype by IHC | | | | | | | | | |
| Luminal B (HER2−) | 0.55 | [0.267, 1.13] | 0.11 | 1.38 | [0.912, 2.1] | 0.13 | 1.33 | [0.86, 2.05] | 0.20 |
| Triple Negative | 0.595 | [0.16, 2.22] | 0.44 | 1.36 | [0.712, 2.59] | 0.35 | 1.28 | [0.608, 2.7] | 0.52 |
| HER2+ | 1.04 | [0.351, 3.06] | 0.95 | 1.52 | [0.754, 3.08] | 0.24 | 1.32 | [0.621, 2.8] | 0.47 |
| Grade | | | | | | | | | |
| Grade 2 | 1.71 | [0.665, 4.39] | 0.27 | 1.86 | [0.895, 3.87] | 0.10 | 1.78 | [0.848, 3.72] | 0.13 |
| Grade 3 | 2.06 | [0.661, 6.43] | 0.21 | 2.31 | [1.07, 4.96] | 0.03 | 1.92 | [0.833, 4.43] | 0.13 |
| Systemic Therapy | 0.451 | [0.103, 1.97] | 0.29 | 0.398 | [0.162, 0.975] | 0.04 | 0.352 | [0.141, 0.874] | 0.02 |

TABLE 5
Exemplary genomic classifier interaction analysis.

| | | |
|---|---|---|
| Interaction New | | |
| Classifier and RT | UVA HR | UVA P |

| | | | |
|---|---|---|---|
| New classifier | 1.89 | [1.04, 3.46] | 0.04 |
| RT | 0.0632 | [0.0142, 0.282] | 0.0003 |
| New classifier:RT | 3.86 | [1.41, 10.5] | 0.008 |

| Interaction New Classifier and RT, | | | |
|---|---|---|---|
| adjusted for clinical variables | MVA HR | | MVA P |
| New Classifier | 1.81 | [0.985, 3.34] | 0.06 |
| RT | 0.0638 | [0.0142, 0.287] | 0.0003 |
| Subtype by IHC | Luminal A as Reference | | |
| Luminal B (HER2−) | 1.05 | [0.729, 1.52] | 0.78 |
| Triple Negative | 0.986 | [0.519, 1.88] | 0.97 |
| HER2+ | 1.24 | [0.675, 2.29] | 0.49 |
| Grade | Grade 1 as Reference | | |
| Grade 2 | 1.84 | [1.03, 3.29] | 0.04 |
| Grade 3 | 2 | [1.03, 3.9] | 0.04 |
| Systemic Therapy | 0.385 | [0.178, 0.833] | 0.02 |
| New classifier:RT | 3.85 | [1.41, 10.5] | 0.009 |

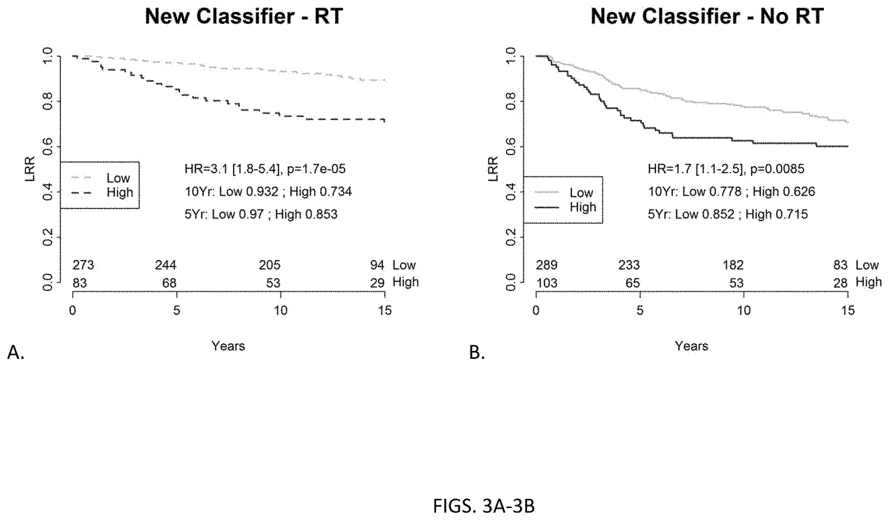

The new signature was highly prognostic in the patients who received RT (HR=3.1 [1.8-5.5], p<0.001), and for patients who did not receive RT (HR=1.7 [1.1-2.5], p=0.009) in univariate analysis (FIG. 3 and Table 4). When accounting for systemic therapy, subtype, and grade in multivariable analysis, the signature remains highly prognostic in patients treated with RT (HR=3.1[1.8-5.5]), p<0.001 and to patients not treated with RT (HR=1.6 [1.1-2.4], p=0.02) (Table 4).

Exemplary Genomic Classifier Performance is Better than Age or Genomic Model Independently

Performance of the classifier was compared to age and the genomics component individually as follows. Age is both prognostic (HR=0.97 [0.96-0.99], p=0.0009) and predictive (p=0.04) for RT benefit in the SweBCG91RT cohort (Table 6). The genomic portion of the model, evaluated as a model of 27 genes, is also prognostic (HR=3.1[1.5-6.5], p=0.002) and predictive (p=0.02). The inclusion of age and genomic portion of the model in a multivariable Cox regression model finds that both are statistically significant to the locoregional recurrence endpoint (age p=0.003, genomic p<0.001), indicating that the two provide independent information from each other. The new classifier, which combines age and genomic variables, is better at stratifying for LRR risk than the components separately.

TABLE 6
Age and Genomics, LRR endpoint.

| | UVA HR | | UVA P | MVA HR | | MVA P |
|---|---|---|---|---|---|---|
| Interaction with Age | | | | | | |
| Age | 0.97 | [0.96-0.99] | 0.0009 | 0.983 | [0.965, 1] | 0.0794 |
| RT | 0.43 | [0.31-0.60] | 6.00E−07 | 3.66 | [0.47, 28.5] | 0.216 |

| | | | | | |
|---|---|---|---|---|---|
| Age:RT | na | na | 0.962 | [0.927, 0.999] | 0.0415 |

| | | | | | | |
|---|---|---|---|---|---|---|
| Interaction with Genomic-only | | | | | | |
| model (LRR27) | | | | | | |
| LRR27 | 3.1 | [1.5-6.5] | 0.002 | 1.62 | [0.658, 4.01] | 0.293 |
| RT | 0.43 | [0.31-0.60] | 6.00E−07 | 0.783 | [0.44, 1.39] | 0.407 |
| LRR27:RT | | | | 6.27 | [1.31, 29.9] | 0.0213 |

| Multivariable model with | All | | | RT | | | No RT | | |
|---|---|---|---|---|---|---|---|---|---|
| clinical variables | HR | | P | HR | | P | HR | | P |
| Age | 0.972 | [0.956, 0.988] | 6.25E−04 | 0.949 | [0.918, 0.982] | 2.23E−03 | 0.982 | [0.963, 1] | 0.06 |
| LRR27 | 2.59 | [1.21, 5.54] | 0.01 | 6.79 | [1.81, 25.5] | 4.54E−03 | 1.51 | [0.586, 3.9] | 0.39 |
| Subtype by IHC | Luminal A as Reference | | | | | | | | |
| Luminal B (HER2−) | 0.997 | [0.69, 1.44] | 0.99 | 0.567 | [0.276, 1.16] | 0.12 | 1.36 | [0.88, 2.1] | 0.17 |
| Triple Negative | 0.933 | [0.492, 1.77] | 0.83 | 0.507 | [0.136, 1.88] | 0.31 | 1.29 | [0.613, 2.73] | 0.50 |
| HER2+ | 1.13 | [0.614, 2.09] | 0.69 | 0.961 | [0.328, 2.81] | 0.94 | 1.41 | [0.654, 3.06] | 0.38 |
| Grade | Grade 1 as Reference | | | | | | | | |
| Grade 2 | 1.96 | [1.1, 3.51] | 0.02 | 1.77 | [0.687, 4.56] | 0.24 | 1.87 | [0.888, 3.92] | 0.10 |
| Grade 3 | 2.28 | [1.17, 4.44] | 0.02 | 2.06 | [0.651, 6.52] | 0.22 | 1.97 | [0.852, 4.57] | 0.11 |
| Systemic Therapy | 0.409 | [0.189, 0.887] | 0.02 | 0.433 | [0.0977, 1.92] | 0.27 | 0.354 | [0.142, 0.883] | 0.03 |

### Discussion

Multiple genomic signatures were examined for prognostication of LRR and interaction with RT from a large randomized phase III clinical study for +/−RT. Some of the previous signatures intended for locoregional recurrence prognostication and RT treatment prediction are prognostic, but none are predictive for benefit from RT. A novel clinical-genomic classifier was developed in independent public data and validated to be highly prognostic for locoregional recurrence, as well as significantly treatment predictive for adjuvant RT. These results showed that methods and classifiers of the present invention are prognostic for locoregional recurrence and identify breast cancer patients who respond to adjuvant radiotherapy (radiation treatment). These results further showed that the methods and classifiers of the present are useful for treating breast cancer.

The genomic classifier of the present invention identified patients at high risk of LRR and that are resistant to RT, thus identifying them as candidates for intensified treatment such as upfront mastectomy. Further, the genomic classifier of the present invention showed that breast cancer patients of highest risk of LRR have the lowest effect of RT. Thus, these results showed that the methods and classifiers of the present invention are useful for identifying subjects that should be treated with mastectomy, radiation boost, or adjuvant systemic therapy.

All references cited herein are hereby incorporated by reference herein in their entirety.

1. A method for prognosing and/or predicting benefit from adjuvant radiotherapy in a subject having breast cancer, the method comprising:
a) obtaining or having obtained an expression level in a sample from a subject for a plurality of genes, wherein the plurality of genes are selected from Table 2; and
b) determining that the subject is at low risk or not at low risk of cancer recurrence based on the expression level, and/or likely to benefit from adjuvant radiotherapy based on the expression level, thereby prognosing and/or predicting benefit from adjuvant radiotherapy in the subject.

2. The method of claim 1, further comprising: administering adjuvant radiotherapy therapy if the subject is identified as likely to benefit from adjuvant radiotherapy, and recommending treatment intensification if the subject is not identified as likely to benefit from adjuvant radiotherapy.

3. The method of claim 1, wherein the cancer recurrence is local or locoregional recurrence or distant recurrence (metastasis).

4. The method of claim 1, further comprising determining that the subject is at low risk of cancer recurrence based on the age of the subject, or determining that the subject is not at low risk of cancer recurrence based on the age of the subject.

5. The method of claim 1, further comprising calculating a risk score for the subject, wherein adjuvant radiotherapy is administered to the subject if the subject is identified as being as likely to benefit from adjuvant radiotherapy based on both the risk score and the expression levels of the genes selected from Table 2 in the biological sample, and recommending treatment intensification if the subject is not identified as being likely to benefit from adjuvant radiotherapy based on both the risk score and the expression levels of the genes selected from Table 2 in the biological sample.

6. The method of claim 1, wherein the plurality of genes are selected from the group consisting of ATP binding cassette subfamily C member 8 (ABCC8), BTG anti-proliferation factor 3 (BTG3), cyclin B1 (CCNB1), centromere protein F (CENPF), creatine kinase mitochondrial 1B (CKMT1B), cornichon family AMPA receptor auxiliary protein 4 (CNIH4), cellular retinoic acid binding protein 2 (CRABP2), enoyl-CoA hydratase domain containing 2 (ECHDC2), eukaryotic elongation factor 2 kinase (EEF2K), eukaryotic translation initiation factor 3 subunit L (EIF3L), ectonucleoside triphosphate diphosphohydrolase 6 (putative) (ENTPD6), epoxide hydrolase 2 (EPHX2), H2A histone family member Z (H2AFZ), hydroxysteroid 17-beta dehydrogenase 4 (HSD17B4), karyopherin subunit alpha 2 (KPNA2), lymphoid enhancer binding factor 1 (LEF1), NEDD4 binding protein 2 like 1 (N4BP2L1), NIMA related kinase 10 (NEK10), prefoldin subunit 4 (PFDN4), pleckstrin and Sec7 domain containing 3 (PSD3), runt related transcription factor 1 (RUNX1), SEC14 like lipid binding 2 (SEC14L2), serine/threonine kinase 39 (STK39), TBC1 domain family member 8 (TBC1D8), thymosin beta 15a (TMSB15A), USO1 vesicle transport factor (USO1), and zinc finger and BTB domain containing 16 (ZBTB16).

7. The method of claim 1, wherein the biological sample is a biopsy or a tumor sample.

8. (canceled)

9. The method of claim 1, further comprising treating the subject with mastectomy, radiation boost, or adjuvant systemic therapy.

10. The method of claim 1, wherein the subject is treated with radiotherapy following breast conserving surgery (BCS).

11. The method of claim 1, wherein the subject has estrogen receptor positive (ER+) breast cancer, human epidermal growth factor receptor 2 negative (HER2-) breast cancer, Stage I-II breast cancer, or node negative breast cancer and/or is post-menopausal.

12. The method of claim 1, wherein the expression levels of all of the genes selected from Table 2 are measured in the biological sample.

13. The method of claim 1, wherein the levels of expression of one or more of the genes selected from Table 2 are increased or reduced compared to a control.

14. The method of claim 1, wherein said measuring the levels of expression comprises performing in situ hybridization, a PCR-based method, an array-based method, an immunohistochemical method, an RNA assay method, or an immunoassay method.

15. (canceled)

16. The method of claim 1, wherein said measuring the level of expression comprises measuring the level of an RNA transcript.

17. The method of claim 1, wherein the method is performed prior to treatment of the subject with adjuvant chemotherapy, endocrine therapy, or radiotherapy.

18. A kit for determining a prognosis of a subject having breast cancer and whether or not to treat the subject with adjuvant radiotherapy, the kit comprising agents for measuring levels of expression of a plurality of genes selected from Table 2.

19. The kit of claim 18, wherein the kit comprises agents for measuring the levels of expression of all of the genes listed in Table 2.

20. The kit of claim 18, wherein said agents comprise reagents for performing in situ hybridization, a PCR-based method, an array-based method, an immunohistochemical method, an RNA assay method, or an immunoassay method.

21. The kit of claim 18, wherein said agents comprise one or more of a microarray, a nucleic acid probe, a nucleic acid primer, or an antibody.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)
